(19) United States
(12) Patent Application Publication
(10) Pub. No.: US 2002/0019573 A1
(43) Pub. Date: Feb. 14, 2002

(54) Process for the preparation of d,l-menthol

(76) Inventors: Andreas Schlemenat, Krefeld (DE); Reinhard Langer, Tonisvorst (DE); Claus Dreisbach, Koln (DE); Hans-Jurgen Gross, Duisburg (DE); Thomas Prinz, Leverkusen (DE); Andreas Schulze-Tilling, League City, TX (US); Michael Friederich, Krefeld (DE); Jorg-Dietrich Jentsch, Krefeld (DE); Gerald John, Dusseldorf (DE)

Correspondence Address: DIDERICO VAN EYL, Bayer Corporation, 100 Bayer Road, Pittsburgh, PA, 15205, US

(21) Appl. No.: 09/850,971

(22) Filed: May 8, 2001

(30) Foreign Application Priority Data
May 12, 2000 (DE) 10023283.3

(51) Int. Cl. C07C 35/12

(52) U.S. Cl. 568/829

(57) ABSTRACT

Process for the preparation of d,l-menthol by catalytic isomerization of stereoisomers of menthol or mixtures of these isomers at temperatures of from 30 to 170° C. in the presence of a supported ruthenium catalyst, the support material used being Al₂O₃.

### BACKGROUND

The present invention relates to a novel process for the preparation of d,l-menthol by rearrangement of stereoisomers of menthol over supported ruthenium-containing catalysts at temperatures of from 30 to 170° C.

Of the naturally occurring cyclic terpene alcohols, l-menthol, the major constituent of peppermint oil, occupies a special position because of its cooling and refreshing action. l-Menthol is therefore used as a fragrance or flavoring and is used in the drug industry.

The preparation of menthol by catalytic hydrogenation of compounds which have the carbon backbone of menthane with at least one double bond and are substituted in the 3-position by oxygen (such as e.g. thymol) leads to a mixture of the eight optically active menthols: d,l-menthol, d,l-isomenthol, d,l-neomenthol and d,l-neoisomenthol. These eight optically active menthols differ with regard to their organoleptic properties. Only l-menthol has the characteristic peppermint odor and the refreshing action already mentioned. It is therefore the most valuable of the menthol stereoisomers. Endeavours have therefore been to carry out the hydrogenation such that the largest amount possible of d,l-menthol forms. Separation of the mixture of the eight stereoisomeric menthols then gives the d,l-menthol as racemate, which can then be resolved to give the antipodes.

The boiling points of d,l-isomenthol (218.6° C. at 760 torr (1.01 bar); 75-78° C. at 2.5 torr (3.3 mbar)) and d,l-menthol (216.5° C. at 760 torr (1.01 bar); 75-78° C. at 2.5 torr (3.3 mbar)) are very close to one another. The separation efficiency of a column during distillative separation of the individual menthol isomers is therefore determined in particular by the ratio of menthol to isomenthol. For a high space-time yield of d,l-menthol in the distillative separation, it is therefore necessary that not only the menthol content in the mixture to be separated is as high as possible, but also that the isomenthol content is as low as possible. The yield of menthol is thus essentially determined, for a given distillation column, by the starting ratio of menthol to isomenthol.

For the preparation of d,l-menthol compounds, it is known to hydrogenate compounds which have the carbon backbone of menthane with at least one double bond and which are substituted in the 3-position by oxygen, for example thymol, with hydrogen in continuous processes over solid catalyst beds, or to rearrange stereoisomers of menthol over solid catalyst beds.

DE 2 314 813 A 1 describes a process for the hydrogenation of compounds which have the carbon backbone of menthane with at least one double bond and which are substituted in the 3-position by oxygen, over a bed of a cobalt/manganese catalyst at temperatures of from 170° C. to 220° C. and pressures above 25 bar, preferably above 200 bar. In the examples, the processes are carried out at temperatures of from 180° C. to 210° C. and at pressures above 200 bar, giving a mixture of the eight stereoisomeric menthols which consists of 59.5 to 59.9% of the racemic d,l-menthol and of 10.6 to 10.8% of d,l-isomenthol. The menthol/isomenthol ratio is at most 5.7. Modification of this catalyst with copper gave menthol mixtures with d,l-menthol contents of 57.6% and d,l-isomenthol contents of 9.2% (menthol/isomenthol ratio 6.3), in which, however, 4 to 5% of undesired by-products in the form of non-reusable hydrocarbons were present.

EP 0 563 611 A 1 and DE 197 18 116 A 1 discloses that the hydrogenation of aromatic or partially hydrogenated cyclic compounds which have the carbon backbone of menthane with at least one C═C double bond and which are substituted in the 3-position by oxygen with hydrogen can be carried out over a fixed-bed catalyst which, on a support which has been doped with a rare earth (re) metal and manganese, comprises palladium, ruthenium or rhodium or a mixture of these elements as active constituents and alkali metal hydroxides and/or sulfates as promoters. In the examples, the processes were carried out at temperatures of from 180 to 240° C. and pressures of from 270 to 300 bar, giving menthol mixtures which comprised about 52 to 57% d,l-menthol. 11.5 to 14.8% of d,l-isomenthol were formed (menthol/isomenthol ratio 3.6 to 4.4).

EP 743 296 A 1 describes catalysts which consist of support-free, compressed powders of cobalt, manganese and alkaline earth metal oxides or hydroxides and operate at temperatures of from 150° C. to 230° C. and pressures of from 25 to 350 bar. In the examples given, the processes are carried out at temperatures above 165° C. and at pressures of more than 200 bar. There is no discussion of the composition of the resulting menthol mixtures.

The rearrangement of stereoisomers of l-menthol is described in U.S. Pat. No. 5,756,864 at temperatures of from 200 to 350° C. and hydrogen pressures of from 50 to 350 bar, preferably 100 to 300 bar, d-menthol is racemized and isomerized in a continuous process over a catalyst which consists of support-free, pressed powders of nickel, manganese and alkaline earth metal hydroxides or oxides. This gave menthol mixtures which consisted of at most 59.8% of d,l-menthol.

All of the hitherto described processes for the preparation of menthol by hydrogenation starting from compounds having the carbon backbone of menthane or isomerization of stereoisomers of menthol are carried out at high pressures and high temperatures. In none of the processes described are more than 59.9% of d,l-menthol formed. The isomenthol content is, as a minimum, about 10 to 11%.

U.S. Pat. No. 2,843,636 describes the isomerization of stereo-isomers of menthol to d,l menthol using hydrogen in the presence of a hydrogenation catalyst from the group copper chromite, cobalt and nickel at 260 to 280° C. and 500 to 1300 p.s.i.g. (34 to 90 bar) in autoclaves. As well as about 10 to 12% d,l-isomenthol, the resulting mixtures had a d,l-menthol content of from 60 to 64%. However, in the low-pressure process described, approximately 5% of non-reusable hydrocarbons are produced as by-products, presumably because of the very high temperature.

German Patent Application 198 53 562.7 describes a low-pressure hydrogenation of thymol over a stationary catalyst bed, which has a temperature gradient: the first two of five reactor tubes connected in series are heated to 180° C., and the back three reactor tubes to 80 to 90° C. Using a catalyst which, on a support which has been doped with a rare earth (re) metal and with manganese, comprises ruthenium as active constituent and alkaline metal hydroxides as promoters, it was possible, at a pressure of 3 bar, to obtain a menthol isomer mixture which comprised 64.4% of menthol and 12.1% of isomenthol (menthol/isomenthol ratio is 5.3). The isomerization of a hydrogen-saturated mixture of d,l-neomenthol, d,l-isomenthol and d,l-menthol produced, at atmospheric pressure, an isomer mixture having a composition of 65.3% of d,l-menthol and 12.1% of isomenthol. In this low-pressure process it is possible to achieve high menthol contents of about 65%. The menthol/isomenthol ratio is at most 5.4.

The higher the proportion of d,l-menthol and the higher the menthol/isomenthol ratio, the easier it is to work up the isomer mixtures by distillation to give pure d,l-menthol. It was therefore an object of the invention to find a selective and technically simple process for the preparation of d,l-menthol which, using an easy-to-prepare catalyst, permits d,l-menthol contents of ≧60% and menthol/isomenthol ratios of ≧6.0 in the product mixture, and as far as possible avoids the formation of undesired by-products. The percentages given are understood here as meaning area percentages which arise during the gas chromatographic analysis of the product mixture. Menthol/isomenthol ratio is understood as meaning the ratio of the area per cent (GC) of menthol to the area per cent (GC) of isomenthol.

### DESCRIPTION

The invention relates to a process for preparing a d,l-menthol comprising catalytic isomerizing stereoisomers of menthol or mixtures of these isomers at a temperature that ranges from about 30 to about 170° C. in the presence of a supported ruthenium catalyst, wherein the support material is Al₂O₃. These and other features, aspects, and advantages of the present invention will become better understood with reference to the following description and appended claims.

We have now found that, starting from the isomers of menthol or from mixtures of these isomers which have a d,l-menthol content of from 0 to 60%, typically of about 55%, it is possible, by isomerization at temperatures of from 30 to 170° C. using simple supported ruthenium catalysts (heterogeneous catalysis), to prepare menthol-richer mixtures which have d,l-menthol contents of, for example, up to 67.3%, isomenthol contents of, for example, only 8.2% and menthol/isomenthol ratios of >6.0, for example of up to 8.1. It has also been found that the supported ruthenium catalysts can be regenerated/reactivated during operation by adding bases.

The invention is surprising in as much as, using easy-to-prepare catalysts at low temperatures and pressures, it is also possible to achieve an isomerization of isomenthol. Surprisingly, it has been found that even at low partial hydrogen pressures, in particular in the absence of hydrogen, a very high isomerization rate of isomenthol is observed, although the reciprocal rearrangement of the individual menthols takes place via a dehydrogenation/hydrogenation.

Accordingly, the invention provides a process for the preparation of d,l-menthol by catalytic isomerization of stereoisomers of menthol or mixtures of these isomers at temperatures of from 30 to 170° C., the isomerization being carried out in the presence of a supported ruthenium catalyst and the support material used being Al₂O₃.

The starting material which may be used in the process according to the invention is the individual isomers of menthol (isomenthol, neomenthol, neoisomenthol) or mixtures of these isomers, it being possible for the starting material to already comprise menthol. It is possible, for example, to use isomer mixtures produced during the racemization of optically active menthols or which are left behind during the distillative separation of d,l-menthol from an isomer mixture. It is also possible, for example, to use the menthol isomer mixtures which arise during the hydrogenation of cyclic compounds which have the carbon backbone of menthane with at least one double bond and which are substituted in the 3-position by oxygen (e.g. thymol, menthone, isomenthone). Here, the reactor in which the isomerization according to the invention is carried out can be connected downstream, for example, of a reactor for the hydrogenation of cyclic compounds which have the carbon backbone of menthane with at least one double bond and which are substituted in 3-position by oxygen, or a reactor for the racemization/ isomerization of d-menthol or other isomers of l-menthol or a, for example, distillative separation of a mixture of the isomers of menthol. The isomerization reactor for the process according to the invention can, for example, also be connected between an existing hydrogenation, isomerization or racemization reactor and a subsequent e.g. distillative separation. Where appropriate, it is possible to use mixtures of different product streams (e.g. from hydrogenation and separation) as starting material in the isomerization process according to the invention. The reactor for the isomerization can, however, also be operated on its own, where appropriate.

The d,l-menthol-containing isomer mixture prepared by the process according to the invention can be separated for the isolation of pure d,l-menthol, for example by distillation.

The process according to the invention can be carried out discontinuously or continuously, e.g. in a stirred-tank reactor, as a trickle phase, in the liquid phase with suspended catalyst, as bubble column or over a stationary catalyst bed. Preference is given to carrying out the process according to the invention in the liquid phase in reactors with stationary catalyst beds.

The process according to the invention can be carried out in the presence of solvents. However, preference is given to a solvent-free implementation.

The catalytic isomerization can be carried out in the process according to the invention, for example, without the addition of hydrogen under reduced pressure (e.g. at a pressure of down to about 50 mbar), at atmospheric pressure (1 bar) or at increased pressure up to 300 bar, preferably at atmospheric pressure to about 50 bar, very particularly preferably at atmospheric pressure to about 10 bar. The process according to the invention can also be operated in the presence of hydrogen, here, the liquid phase is saturated with hydrogen prior to entry into the reactor, a hydrogen-saturated starting material stream (e.g. an isomer mixture from a hydrogenation reactor) is used, or gaseous hydrogen is passed into the reactor together with the starting material such that a partial hydrogen pressure between about 0.001 and about 300 bar, preferably between about 0.01 and about 50 bar, particularly preferably between about 0.01 and about 10 bar, is established. However, preference is given to using no additional hydrogen in the isomerization according to the invention, meaning that only the hydrogen which may have been introduced into the isomerization reactor via the starting materials used is present.

The process according to the invention is carried out at temperatures of from about 30 to about 170° C., preferably at temperatures of from about 50 to about 150° C., particularly preferably at temperatures of from about 70 to about 140° C.

For the process according to the invention, supported ruthenium catalysts are used (heterogeneous catalysis). The supported ruthenium catalysts preferably comprise from about 0.1 to about 15 per cent by weight, particularly preferably from about 2 to about 9 per cent by weight, of ruthenium. Where appropriate, use is made of supported ruthenium catalysts which, in addition to ruthenium, comprise from about 0.1 to about 10 per cent by weight of one or more further metals from transition group 8 of the Periodic Table of the Elements (Fe, Co, Ni, Rh, Pd, Os, Ir, Pt) and/or Sn and/or Zn, preferably Pt, Sn and/or Zn. The percentages by weight given are based in each case on the weight of the support material.

Starting compounds for the preparation of the catalysts according to the invention are therefore compounds of noble metals of transition group 8 of the Periodic Table of the Elements, tin or zinc. Examples which may be mentioned are the halides, nitrates, acetates, organic complexes with acetylacetone or amino acids.

The support material used is Al₂O₃ in the various modifications, preferably in the γ-modification. The aluminium oxide used as support material advantageously has a BET surface area of about ≧100 m²/g, preferably about ≧160 m²/g, particularly preferably about ≧180 m²/g. Particular preference is given to aluminium oxide which additionally has a high proportion of macroporous pores (about >50 nm) and has a pore volume of about ≧300 mm³/g, preferably about ≧600 mm³/g. Suitable support materials which may be mentioned by way of example are the commercially available Al₂O₃ supports SPH 1515, SPH 531, SPH 501 from Rhodia, D 10-10 from BASF and SA 6176 from Norton.

The catalyst support can, for example, be used in the form of powders having particle sizes of from about 0.001 to about 0.1 mm, crushed and screened material having particle sizes between about 0.05 and about 5 mm, or in moldings, such as extrudates, pellets, spheres or granulates having diameters of from about 0.2 to about 30 mm.

For the preparation of the catalysts it is possible, for example, for the procedure to involve applying firstly ruthenium and optionally one or more further metals from transition group 8 of the Periodic Table of the Elements and/or tin and/or zinc to one of said support materials. The application can be carried out by treating, for example impregnating or spraying, the support material with solutions of the metals. For this purpose, use is made, for example, of the chlorides, acetates and/or nitrates. This application of the metals can be carried out in one step with dissolved mixtures of the salts or successively with the solutions of the individual compounds. After each application, the catalyst may be dried.

A catalyst prepared in said manner is reduced, for example, by treatment with hydrogen or hydrogen/nitrogen mixtures with a hydrogen content of more than about 1% at a temperature of from about 20 to about 400° C., preferably from about 30 to about 250° C. The reduction can also be carried out with other reducing agents, such as, for example, hydrazine. The reduced catalyst is then advantageously washed free from chloride and/or nitrate.

The applied metal can, for example, also be fixed to the support by treating the support impregnated with ruthenium and optionally further metals with a solution of basic salts, e.g., alkali metal or alkaline earth metal hydroxides or oxides, such as e.g., NaOH, KOH, the metal precipitating out as oxide or hydroxide. If the metal is fixed to the support, the reduction and the washing out of soluble constituents can be carried out in any order. Preferably, the soluble constituents are firstly washed out and then the catalyst is reduced. The reduction can also be carried out in situ in the reactor in which the isomerization according to the invention is to be carried out.

After each impregnation step with metal salt solution or basic salts or after washings, a drying step may be carried out. It is, however, also possible to use the support in the next preparation step without drying.

The space velocity in the process according to the invention is, for example, from about 0.005 to about 5 kg of starting material per liter of catalyst and per hour, preferably from about 0.03 to about 2 kg/l·h, particularly preferably from about 0.06 to about 1.0 kg/l·h. The space-time yield of the isomerization according to the invention increases with increasing space velocity. On the other hand, the proportion of d,l-menthol in the product mixture decreases and the menthol/isomenthol ratio drops, the degree of the reduction depending heavily on the reaction temperature chosen. The maximum space velocity at a given reaction temperature at which the product mixture has about ≧60% d,l-menthol and a menthol/isomenthol ratio of about ≧6.0 can be readily determined by the person skilled in the art.

The process according to the invention scarcely leads to the formation of non-usable by-products, such as undesired hydrocarbons. The resulting reaction mixtures comprise a high content of d,l-menthol, preferably ≧60%, particularly preferably about ≧64%, a low proportion of d,l-isomenthol, for example from about 8.2% to about 11%, preferably from about 9% to about 10% and a high menthol/isomenthol ratio, preferably from about 6.0 to about 8.2, particularly preferably from about 6.5 to about 7.5, very particularly preferably from about 6.8 to about 7.3, meaning that the desired product can be separated off easily, e.g. by rectification/distillation.

The catalyst has long service lives (for example >6000 hours), within which only slight deactivation is observed. The catalyst can be regenerated/reactivated by adding small amounts of bases, such as e.g. alkoxides, oxides or hydroxides of the alkali or alkaline earth metals (e.g. KOtBu, KOH, NaOH).The base can be added by the action of a solution of a base following removal of the catalyst or within the reactor itself. Preferably, the base is added to the menthol starting material in a continuous operation by adding a basic solution to the menthol or, without using a solvent, dissolving the base itself in the menthol.

### EXAMPLES

The process according to the invention is further illustrated below by examples, the process according to the invention not being limited to the examples, and the latter accordingly not being regarded as limiting. Unless stated otherwise, percentages are area percentages from a gas chromatographic (GC) analysis.

### Example 1

### Preparation of a Supported Ru Catalyst (6% by Weight of Ru)

1000 g (about 2.24 l) of a commercially available γ-Al₂O₃ having a BET surface area of about 255 m²/g (SA 6176 from Norton, extrudate with particle diameter 1/16″ (about. 1.6 mm), bulk density about. 0.44 kg/l) were initially introduced into a large rotary evaporator (10 l flask), an aqueous solution of 300 g of ruthenium(lII) chloride (commercially available solution containing 20% by weight of Ru, 60 g of Ru) in 1153 g of distilled water is added, and the mixture is rotated for 10 minutes (about 16 rpm (revolutions per minute)). The solvent was distilled off at 90° C. and 10 mbar. The catalyst was reduced in a stream of hydrogen at 250° C. The catalyst was then washed with distilled water until the wash water was chloride-free. The catalyst was then dried in a rotary evaporator (90° C., 10 mbar).

### Example 2

### Experimental Plant

The experimental plant consisted of 5 oil-bath-thermostated double-walled reactor tubes each with a length of 1 m and an internal diameter of 15 mm, which were connected in series one above the other. A sampling point was located behind each tubular reactor. The reactors were heated by two thermostated baths. The reactor tubes were each filled with about 129 ml of the catalyst from Example 1 (bulk height in each tube about 80 cm, total amount 643 ml or 284 g). The menthol isomer mixture used was conveyed into the tubular reactor using a membrane pump. The starting material could, if desired, be passed through the tubular reactors from above (trickle phase) or from below (liquid phase). The hydrogen could be added by saturating the starting material or by passing hydrogen through the reactor tubes from above or below in a certain amount (trickle phase, bubble column). In the experimental plant, operating pressures from atmospheric pressure to 30 bar above atmospheric were possible. In the pressurized procedure (bubble column, trickle phase), hydrogen was conveyed into the plant via a pressure reducer set to the desired pressure. The pressure in the plant was kept constant by adjusting the amount of offgas via needle valves to a certain amount (rotameter). The product was discharged in a level-controlled product separator (2 liters). If the reaction was carried out without hydrogen (liquid phase, trickle phase), the gas required to establish the pressure (for example nitrogen) was not added and discharged again until directly before the product separator. It thus does not flow through the reactor.

### Example 3

### Preparation of d,l-menthol

The reactor tubes of the experiment plant from Example 2 were heated to 100 to 130° C. A menthol isomer mixture was pumped into the reactor from below in a liquid-phase reaction at space velocities between 0.07 and 0.75 kg (starting material)/l (catalyst)·h and at a pressure of 2 bar above atmospheric. Hydrogen was not added. Table 1 gives the starting material composition and the composition of the product for the individual temperatures and space velocities.

TABLE 1
Composition of the product as a function of temperature and space velocity; catalyst 6% by weight of Ru on Al₂O₃,
space velocity in kg (starting material)/l(catalyst) · h

| Temperature | Space velocity | Menthol | Neomenthol | Isomenthol | Neoiso- | Menthones | Hydrocarbons | Menthol/ |
|---|---|---|---|---|---|---|---|---|
| [° C.] | [kg/l*h] | [%] | [%] | [%] | menthol [%] | (total) [%] | [%] | Isomenthol |
| Starting | | 55.8 | 28.5 | 12.2 | 2.20 | 0.34 | 0.99 | 4.57 |
| material | | | | | | | | |
| 100 | 0.070 | 65.8 | 23.1 | 9.1 | 0.76 | 0.28 | 0.87 | 7.23 |
| 100 | 0.095 | 65.0 | 23.4 | 9.3 | 0.83 | 0.61 | 0.87 | 7.01 |
| 100 | 0.130 | 65.2 | 23.0 | 9.7 | 0.79 | 0.41 | 0.87 | 6.69 |
| 100 | 0.161 | 65.2 | 22.9 | 10.0 | 0.82 | 0.31 | 0.85 | 6.55 |
| 100 | 0.197 | 64.6 | 22.8 | 10.3 | 0.85 | 0.39 | 1.04 | 6.27 |
| 110 | 0.161 | 64.6 | 23.8 | 9.4 | 0.85 | 0.39 | 1.05 | 6.88 |
| 110 | 0.231 | 64.2 | 23.7 | 9.7 | 0.87 | 0.40 | 1.12 | 6.64 |
| 110 | 0.296 | 63.7 | 23.8 | 10.1 | 0.90 | 0.42 | 1.13 | 6.34 |
| 120 | 0.299 | 63.2 | 24.5 | 9.7 | 0.99 | 0.64 | 0.91 | 6.52 |
| 120 | 0.419 | 63.1 | 24.5 | 9.9 | 0.99 | 0.53 | 0.95 | 6.34 |
| 120 | 0.592 | 62.4 | 24.7 | 10.3 | 1.03 | 0.58 | 0.97 | 6.03 |
| 130 | 0.750 | 61.7 | 25.2 | 10.0 | 1.11 | 0.90 | 1.00 | 6.15 |

### Example 4

### Preparation of d,l-menthol, Service Life Experiment

The experiment from Example 3 was continued at 100° C. and 2 bar above atmospheric with a space velocity of 0.067 kg (starting material)/l(catalyst)·h. The composition of the resulting isomer mixture is given in Table 2. After a service life of more than 5300 hours, only slight deactivation was observed.

### Example 5

### Preparation of d,l-menthol

The experiment from Example 4 was continued with a menthol isomer mixture as starting material, which had a lower menthol/isomenthol ratio (3.6). The starting material composition and the composition of the product is given in Table 2.

### Example 6

### Regeneration of the Catalyst

The experiment from Example 5 was continued. To regenerate the supported ruthenium catalyst, 2 g of potassium tert-butoxide were dissolved in 20 g of methanol and added to the menthol isomer mixture in the initial charge of starting material (12 l). The addition of KOtBu was repeated. Following the addition of the base, the catalyst produced product compositions comparable with those at the start of Example 4 (see Table 2 on the following page).

TABLE 2
Product composition of the experiments from Example 4 to Example 6; catalyst 6% by weight of Ru on Al₂O₃,
space velocity 0.067 kg (starting material)/l(catalyst) · h

| | Service | Menthol | Neomenthol | Isomenthol | Neoiso- | Menthones | Hydrocarbons | Menthol/ |
|---|---|---|---|---|---|---|---|---|
| | life [h] | [%] | [%] | [%] | menthol [%] | (total) [%] | [%] | Isomenthol |
| Example 4 | Starting | 56.8 | 28.5 | 12.2 | 2.20 | 0.34 | 0.99 | 4.57 |
| | material | | | | | | | |
| | 1974 | 65.8 | 23.4 | 9.2 | 0.76 | 0.34 | 0.52 | 7.12 |
| | 2866 | 65.7 | 23.3 | 9.5 | 0.77 | 0.31 | 0.40 | 6.90 |
| | 3182 | 65.9 | 23.2 | 9.1 | 0.74 | 0.32 | 0.77 | 7.29 |
| | 3852 | 65.6 | 23.4 | 9.2 | 0.75 | 0.32 | 0.75 | 7.14 |
| | 4404 | 65.6 | 23.3 | 9.3 | 0.75 | 0.31 | 0.72 | 7.08 |
| | 4880 | 65.4 | 23.3 | 9.4 | 0.77 | 0.33 | 0.76 | 6.94 |
| | 5319 | 65.3 | 23.3 | 9.5 | 0.77 | 0.34 | 0.78 | 6.88 |
| Example 5 | Starting | 52.1 | 28.4 | 14.4 | 3.23 | 1.22 | 0.74 | 3.62 |
| | material | | | | | | | |
| | 5369 | 65.3 | 23.3 | 9.5 | 0.77 | 0.35 | 0.80 | 6.89 |
| | 5621 | 64.5 | 23.6 | 9.4 | 0.77 | 1.17 | 0.58 | 6.85 |
| Example 6 | Starting | 56.3 | 27.7 | 12.5 | 2.14 | 0.28 | 1.00 | 4.50 |
| | material | | | | | | | |
| | 5767 | 65.2 | 23.3 | 9.6 | 0.77 | 0.34 | 0.80 | 6.78 |
| Addition of | 6130 | 65.7 | 23.3 | 9.0 | 0.74 | 0.42 | 0.93 | 7.29 |
| KOtBu | 6772 | 65.6 | 23.2 | 9.2 | 0.75 | 0.40 | 0.85 | 7.15 |
| | 7205 | 65.7 | 23.1 | 9.1 | 0.73 | 0.49 | 0.61 | 7.19 |

### Example 7

### Preparation of a Supported Ru Catalyst (4% by Weight of Ru)

2500 g of a commercially available γ-Al₂O₃ having a BET surface area of about 320 m²/g (SPH 501 from Rhodia, spheres, Ø1.4-2.8 mm, bulk density about 0.87 kg/l) were treated as described in Example 1 with a solution of 500 g of ruthenium(III) chloride (commercially available solution containing 20% by weight of Ru, 100 g of Ru) in 1100 g of distilled water. After drying, the catalyst was reduced in a stream of hydrogen at 250° C. and washed until chloride-free.

### Example 8

### Preparation of d,l-menthol

1964 g (about 2250 ml) of the catalyst from Example 7 were introduced into a double-walled glass tubular reactor (length 2.05 m), volume 2.4 liters). The tubular reactor was heated by means of a thermostated oil bath. A menthol isomer mixture was passed through the reactor from below at atmospheric pressure at temperatures of from 90 to 120° C. without the addition of hydrogen (liquid phase). Table 3 gives the composition of starting material and product for a variety of temperatures and space velocities.

TABLE 3
Composition of the product as a function of temperature and space velocity, catalyst 4% by weight of Ru on Al₂O₃,
space velocity in kg (starting material)/l(catalyst) · h

| | | | | | Neoiso- | | | |
|---|---|---|---|---|---|---|---|---|
| Temperature | Space velocity | Menthol | Neomenthol | Isomenthol | menthol | Menthones | Hydrocarbons | Menthol/ |
| [° C.] | [kg/l*h] | [%] | [%] | [%] | [%] | (total) [%] | [%] | Isomenthol |
| Starting | | 55.1 | 29.2 | 12.08 | 2.28 | 0.39 | 0.93 | 4.56 |
| material | | | | | | | | |
| 90 | 0.012 | 67.3 | 22.5 | 8.52 | 0.63 | 0.43 | 0.64 | 7.90 |
| 90 | 0.025 | 67.0 | 22.4 | 8.76 | 0.63 | 0.43 | 0.79 | 7.66 |
| 90 | 0.031 | 66.6 | 22.5 | 8.95 | 0.64 | 0.43 | 0.80 | 7.44 |
| 90 | 0.056 | 64.0 | 23.8 | 10.16 | 0.70 | 0.42 | 0.94 | 6.30 |
| 100 | 0.027 | 66.1 | 22.7 | 8.80 | 0.73 | 0.50 | 1.12 | 7.52 |
| 100 | 0.044 | 65.8 | 23.0 | 8.97 | 0.73 | 0.50 | 0.96 | 7.33 |
| 100 | 0.055 | 65.4 | 23.3 | 9.10 | 0.74 | 0.54 | 0.94 | 7.19 |
| 100 | 0.077 | 64.8 | 23.6 | 9.42 | 0.75 | 0.53 | 0.89 | 6.89 |
| 100 | 0.090 | 64.5 | 23.6 | 9.56 | 0.76 | 0.56 | 0.93 | 6.75 |
| 100 | 0.13 | 63.0 | 24.5 | 10.20 | 0.79 | 0.59 | 0.93 | 6.18 |
| 110 | 0.10 | 64.0 | 24.1 | 9.31 | 0.86 | 0.83 | 0.94 | 6.87 |
| 110 | 0.14 | 63.6 | 24.3 | 9.54 | 0.87 | 0.82 | 0.87 | 6.66 |
| 110 | 0.19 | 63.1 | 24.4 | 9.63 | 0.87 | 0.82 | 1.15 | 6.55 |
| 120 | 0.19 | 62.4 | 24.5 | 9.51 | 0.98 | 1.42 | 1.13 | 6.57 |
| 120 | 0.24 | 62.2 | 24.7 | 9.62 | 0.98 | 1.40 | 1.12 | 6.47 |
| 120 | 0.32 | 61.5 | 25.2 | 9.94 | 0.99 | 1.38 | 1.01 | 6.19 |

### Example 9

### Preparation of an Ru Catalyst (6% by Weight of Ru)

2617.2 g of a commercially available γ-Al₂O₃ having a BET surface area of about 255 m²/g (SA 6176 from Norton, extrudate with particle diameter 1/16″ (1.6 mm), bulk density about 0.44 kg/l) were treated as described in Example 1 with a solution of 785.16 g of ruthenium(II) chloride (commercially available solution containing 20% by weight of Ru, 157.0 g of Ru) in 2329.2 g of distilled water. After drying, to fix the ruthenium, a solution of 559 g of NaOH in 2555 g of distilled water was applied in a second impregnation step, and the catalyst was dried, washed until chloride-free and dried again.

### Example 10

### Preparation of d,l-menthol

5.81 1 (about 2.6 kg) of the catalyst from Example 9 were introduced into a double-walled tubular reactor (volume 7.4 l; length 2.8 m, Ø5.8 cm) which can be operated at pressures between atmospheric pressure and 10 bar. The tubular reactor was heated by means of a thermostated oil-bath. The starting material used could be passed through the tubular reactor as desired from above (trickle phase) or from below (liquid phase). It was possible to additionally pass hydrogen into the reactor from below (bubble column) or from above (trickle phase). The starting material was conveyed into the tubular reactor by means of a membrane pump. The discharge from the tubular reactor was into a level-controlled product separator (2 liters).

For the reduction of the catalyst, the reactor was heated to 150° C., and firstly forming gas (20% by volume of H₂ in N₂), then pure hydrogen, were passed through the reactor. The reactor was cooled to 100° C., and a menthol isomer mixture was passed through the reactor from below at atmospheric pressure without the addition of hydrogen (liquid phase, space velocity 0.09 kg (starting material)/l(catalyst)·h). Table 4 gives the composition of starting material and product.

Table 4 also gives the product composition if hydrogen is added, and if the reaction is carried out under pressure (6 bar) and at lower temperatures.

TABLE 4
Composition of the product as a function of temperature, pressure, space velocity and the addition of
hydrogen; catalyst 6% by weight of Ru on Al₂O₃, space velocity in kg (starting material)/l(catalyst) · h

| | | | | | | Hydro- | |
|---|---|---|---|---|---|---|---|
| Service life | Menthol | Neomenthol | Isomenthol | Neoiso- | Menthones | carbons | Menthol/ |
| [h] | [%] | [%] | [%] | menthol [%] | (total) [%] | [%] | Isomenthol |
| Starting | 56.19 | 27.79 | 12.56 | 2.16 | 0.28 | 1.02 | 4.47 |
| material | | | | | | | |

100° C., Space velocity 0.09 kg/l*h, no hydrogen, atmospheric pressure

| | | | | | | | |
|---|---|---|---|---|---|---|---|
| 37 | 64.92 | 23.27 | 8.83 | 0.75 | 0.97 | 1.26 | 7.35 |
| 157 | 65.12 | 23.32 | 8.86 | 0.75 | 0.81 | 1.13 | 7.35 |
| 781 | 65.34 | 23.24 | 8.77 | 0.75 | 1.24 | 0.66 | 7.45 |

100° C., Space velocity 0.10 kg/l*h, 55 l/h hydrogen through the reactor (bubble column),
atmsopheric pressure

| | | | | | | | |
|---|---|---|---|---|---|---|---|
| 1094 | 65.04 | 23.25 | 9.26 | 0.77 | 0.80 | 0.88 | 7.03 |

100° C., Space velocity 0.10 kg/l*h, no hydrogen, atmospheric pressure

| | | | | | | | |
|---|---|---|---|---|---|---|---|
| 1477 | 65.05 | 23.33 | 8.87 | 0.77 | 1.08 | 0.90 | 7.34 |

100° C., Space velocity 0.11 kg/l*h, no hydrogen (liquid phase), 6 bar above atmospheric

| | | | | | | | |
|---|---|---|---|---|---|---|---|
| 1502 | 65.13 | 23.71 | 9.00 | 0.90 | 0.36 | 0.90 | 7.24 |
| 1622 | 65.27 | 23.62 | 9.06 | 0.90 | 0.28 | 0.88 | 7.20 |

90° C., Space velocity 0.03 kg/l*h, no hydrogen (liquid phase), 6 bar above atmospheric

| | | | | | | | |
|---|---|---|---|---|---|---|---|
| 3016 | 66.77 | 22.38 | 8.64 | 0.60 | 0.93 | 0.49 | 7.73 |

85° C., Space velocity 0.01 kg/l*h, no hydrogen (liquid phase), 6 bar above atmospheric

| | | | | | | | |
|---|---|---|---|---|---|---|---|
| 3170 | 66.81 | 21.73 | 8.20 | 0.62 | 1.80 | 0.59 | 8.15 |
| 3182 | 66.67 | 21.90 | 8.24 | 0.60 | 1.75 | 0.58 | 8.09 |

### Example 11

### Preparation of d,l-menthol

2.24 l (about 0.93 kg) of the catalyst from Example 9 were introduced into the reactor described in Example 8 and, at room temperature, firstly reduced for half an hour with forming gas (10% by volume of H₂ in N₂), then for 5 hours with pure hydrogen (about 60 l/h). The reactor was heated to 100° C. and a menthol isomer mixture was passed through the reactor from below at atmospheric pressure (liquid phase, 100° C.). Table 5 gives the composition of starting material and product.

TABLE 5
Composition of the product as a function of the space velocity; catalyst 6% by weight of Ru on Al₂O₃,
space velocity in kg (starting material)/l(catalyst) · h

| | | | | Neoiso- | | | |
|---|---|---|---|---|---|---|---|
| Space velocity | Menthol | Neomenthol | Isomenthol | menthol | Menthones | Hydrocarbons | Menthol/ |
| [kg/l*h] | [%] | [%] | [%] | [%] | (total) [%] | [%] | Isomenthol |
| Starting | 55.53 | 29.30 | 11.99 | 2.20 | 0.18 | 0.57 | 4.63 |
| material | | | | | | | |

100° C., no hydrogen

| | | | | | | | |
|---|---|---|---|---|---|---|---|
| 0.10 | 64.72 | 23.06 | 8.78 | 0.73 | 1.72 | 0.62 | 7.37 |

100° C., hydrogen saturation of starting material

| | | | | | | | |
|---|---|---|---|---|---|---|---|
| 0.10 | 65.78 | 23.26 | 8.89 | 0.73 | 0.60 | 0.50 | 7.40 |

100° C., no hydrogen

| | | | | | | | |
|---|---|---|---|---|---|---|---|
| 0.11 | 65.56 | 23.20 | 8.98 | 0.74 | 0.67 | 0.59 | 7.30 |
| 0.13 | 65.39 | 23.19 | 9.17 | 0.76 | 0.67 | 0.59 | 7.13 |

### Example 12

### Preparation of a Ru Supported Catalyst (6% by Weight of Ru)

800 g of a commercially available γ-aluminium oxide with a BET surface area of about 255 m²/g (SA 6176 from Norton, extrudate with particle diameter 1/16″ (1,6 mm), bulk density about 0.45 kg/l) are impregnated with a solution of 240 g ruthenium(III) chloride (commercially available solution containing 20% by weight of Ru, 48 g of Ru) in 650 g of distilled water. Then the mixture is covered with a layer of a sodium hydroxide solution (683.5 g NaOH in 2964 g water) and left to stand at room temperature for 20 hours. The catalyst is filtered off, washed with water until free of chloride and then dried in vacuo at 90° C.

### Example 13

### Preparation of d,l-menthol

682 g of the catalyst from Example 12 is filled into the reactor described in Example 8. The catalyst is, at room temperature, firstly reduced for half an hour with forming gas (10% by volume of H₂ in N₂), then for 5 hours with pure hydrogen (about 60 l/h). A menthol isomer mixture was passed through the reactor from below at atmospheric pressure at a temperature of 100° C. without the addition of hydrogen (liquid phase). Table 6 gives the composition of starting material and product.

TABLE 6
Product composition of the experiments from Example 12; catalyst 6% by weight of Ru on Al₂O_{3,}
100° C., space velocity 0.20 kg (starting material)/l(catalyst) · h, no hydrogen

| Service life | Menthol | Neomenthol | Isomenthol | Neoisomenthol | Menthones | Hydrocarbons | Menthol/ |
|---|---|---|---|---|---|---|---|
| [h] | [%] | [%] | [%] | [%] | [%] | [%] | Isomethol |
| Starting | 55.8 | 29.2 | 12.0 | 2.20 | 0.17 | 0.44 | 4.65 |
| material | | | | | | | |
| 519 | 65.8 | 23.1 | 9.25 | 0.75 | 0.45 | 0.45 | 7.11 |
| 1458 | 65.5 | 23.1 | 9.29 | 0.75 | 0.48 | 0.49 | 7.05 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.{PRIVAT }

What is claimed is:

1. A process for preparing a d,l-menthol comprising catalytically isomerizing stereoisomers of menthol or mixtures of these isomers at a temperature that ranges from about 30 to about 170° C. in the presence of a supported ruthenium catalyst, wherein the support material is Al₂O₃.

2. The process according to claim 1, wherein the supported ruthenium catalyst comprises from about 0.1 to about 15 per cent by weight of ruthenium, based on the weight of the support.

3. The process according to claim 1, wherein the supported ruthenium catalyst comprises, in addition to ruthenium, from about 0.1 to about 10 per cent by weight, based on the weight of the support, of one or more further metals from transition Group 8 of the Periodic Table of the Elements and/or tin and/or zinc.

4. The process according to claim 1, wherein the support material comprises aluminium oxide in the γ-modification.

5. Process according to at least one of claim 1, wherein the aluminium oxide used as the support material has a BET surface area that is at least about 100 m²/g.

6. The process according to claim 1, wherein the support material is a macroporous aluminium oxide having a pore volume that is at least about 300 mm³/g.

7. The process according to claim 1, wherein the isomerization is carried out at a pressure that ranges from about 50 mbar to about 300 bar.

8. The process according to claim 1, wherein during the isomerization no additional hydrogen is added.

9. The process according to claim 1, wherein during the isomerization hydrogen having a partial pressure that ranges from about 0.001 to about 300 bar is added.

10. The process according to claim 1, wherein the resulting product has a d,l-menthol content of at least about 60% and a menthol/isomenthol ratio of at least about 6.0.

11. The process according to claim 1, wherein the supported ruthenium catalyst is regenerated by adding a base.
